(19) United States
(12) Patent Application Publication
(10) Pub. No.: US 2022/0047594 A1
(43) Pub. Date: Feb. 17, 2022

(54) METHOD FOR TREATING INTERSTITIAL LUNG DISEASE

(71) Applicant: ALTAVANT SCIENCES GmbH, Basel (CH)

(72) Inventors: Thomas PACK, Durham, NC (US); Magdalena ALONSO-GALICIA, Durham, NC (US)

(73) Assignee: ALTAVANT SCIENCES GmbH, Basel (CH)

(21) Appl. No.: 17/513,491

(22) Filed: Oct. 28, 2021

Related U.S. Application Data
(63) Continuation of application No. 16/714,260, filed on Dec. 13, 2019.
(60) Provisional application No. 62/780,777, filed on Dec. 17, 2018.

(51) Int. Cl. A61K 31/506

(52) U.S. Cl. CPC A61K 31/506

(57) ABSTRACT

There is a method of treating or preventing interstitial lung disease in a patient. The method includes the step of administering to the patient a therapeutically effective amount of the compound of the following formula:

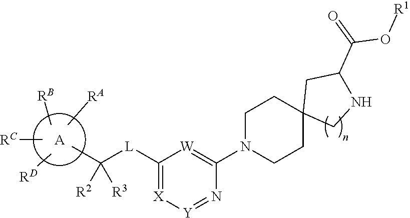

or a pharmaceutically acceptable salt thereof.

### CROSS-REFERENCE TO A RELATED APPLICATION

The present application is a continuation application of U.S. Ser. No. 16/714,260, filed Dec. 13, 2019, which claims priority based on U.S. Provisional Application No. 62/780,777, filed Dec. 17, 2018, both of which are incorporated herein by reference.

### BACKGROUND OF THE DISCLOSURE

### Field of the Disclosure

The present disclosure relates to a method for treating interstitial lung disease (ILD), including pulmonary fibrosis (PF) and idiopathic pulmonary fibrosis (IPF). The present disclosure further relates to a method for treating interstitial lung disease with an inhibitor of tryptophan hydroxylase (TPH1) or with a composition containing same. The present disclosure further relates to a method for treating interstitial lung disease with a therapeutic amount of a THP1 inhibitor.

### Description of the Related Art

Interstitial lung disease (ILD) is a category of diseases that impact the interstitium of the lungs. The interstitium is a lace-like network of tissue that extends throughout both lungs. The interstitium provides support to the lungs' microscopic air sacs (alveoli). Tiny blood vessels travel through the interstitium, allowing gas exchange between blood and the air in the lungs.

ILD causes thickening of the interstitium from inflammation, scarring, and/or extra fluid (edema). Some forms of interstitial lung disease are short-lived, while others are chronic and irreversible. Representative forms include (a) interstitial pneumonia, which is infection by bacteria, viruses, or fungi; (b) pulmonary fibrosis (PF), which is any ILD that results in scarring of the lungs; (c) idiopathic pulmonary fibrosis (IPF), which is chronic, progressive fibrosis (scarring) of the interstitium of unknown cause; (d) nonspecific interstitial pneumonitis: Interstitial lung disease that's often present with autoimmune conditions, such as rheumatoid arthritis or scleroderma; (d) hypersensitivity pneumonitis, which is interstitial lung disease caused by ongoing inhalation of dust, mold, or other irritants; (e) desquamative interstitial pneumonitis, which is partially caused by smoking; and (f) sarcoidosis, which take the form of granulomas usually accompanied by swollen lymph nodes. IPF is the most common form of ILD and PF. PF is a form of ILD.

Idiopathic pulmonary fibrosis (IPF), a form of PF and ILD, is a progressive and fatal lung disease of unknown origin. The disease is characterized by alveolar epithelial cell damage, increased deposition of extracellular matrix in the lung interstitium, enhanced fibroblast/myofibroblast proliferation and activation and, ultimately, distortion of normal lung architecture and loss of respiratory function and lung function. The median survival rate is 3 to 5 years after diagnosis. Symptoms include shortness of breath, a dry cough, and low oxygen levels.

Treatment of IPF to date has taken the form of pharmacological treatment, oxygen supplementation (if hypoxemic), pulmonary rehabilitation, and palliative care to limited effect. Pharmacological treatment options have included pirfenidone and nintedanib. Pirfenidone is and anti-fibrotic, anti-inflammatory and antioxidant pyridine. Nintedanib is an anti-fibrotic, multi-TKR inhibitor. While both are equally effective treatment options, a decision to use either is influenced by tolerance to side effects, notably diarrhea, photosensitivity rash, and liver enzyme increases. Neither pirfenidone nor nintedanib have demonstrated a survival benefit nor they proved to improve the symptoms of these patients. Corticosteroids have been used in treating acute exacerbations. Current treatments for IPF are disclosed in Respiratory Research (2018) 19:32, “Idiopathic pulmonary fibrosis: pathogenesis and management”; J. Clin. Med., 2018, 7, 201, “Idiopathic Pulmonary Fibrosis (IPF): An Overview”; and Am J Respir Crit Care Med, vol 183. pp 788-824, 2011, “An Official ATS/ERS/JRS/ALAT Statement: Idiopathic Pulmonary Fibrosis: Evidence-based Guidelines for Diagnosis and Management”.

Serotonin (5-hydroxytryptamine, 5-HT) is a neurotransmitter that modulates central and peripheral functions by acting on neurons, smooth muscle, and other cell types. 5-HT is involved in the control and modulation of multiple physiological and psychological processes, including in lung and pulmonary diseases. The literature discloses the relationship between 5-HT and pulmonary diseases at PloS One 7, e31617 (2012), “The Role of Circulating Serotonin in the Development of Chronic Obstructive Pulmonary Disease” and Thorax 1999, 54, 161-168, “Role of Serotonin in the Pathogenesis of Acute and Chronic Pulmonary Hypertension”.

Elevated serotonin (5-hydroxytryptamine) levels have been identified as being involved in pulmonary fibrosis. In a bleomycin-induced mouse model, elevated serotonin levels were found in serum, BALF, and the lung. See Eur Respir J 2008; 32: 426-436, “Modulation of bleomycin-induced pulmonary fibrosis by serotonin receptor antagonists in mice”. Sources of elevated lung serotonin include platelets, neuroendocrine cells, mast cells in certain inflammatory and fibrotic conditions, and endothelial cells. Elevated serotonin levels increased expression of lung 5-HT2AR and 2 BR receptor mRNAs. 5-HT2AR and 2 BR antagonists were introduced and were successful in promoting an antifibrotic environment but had minimal effects in reducing lung inflammation. In another bleomycin-induced mouse model, terguride-treated mice showed improved lung function and histology and collagen content and was well-tolerated. See Thorax 2010; 65:949-955, “Increased expression of 5-hydroxytryptamine2A/B receptors in idiopathic pulmonary fibrosis: a rationale for therapeutic intervention”. In another bleomycin-induced mouse model, the presence of serotonin was found to aggravate pulmonary fibrosis in wild-type mice by promoting neutrophil infiltration, inflammation, exudation of proteins and cells, oxidative stress, and upregulation of fibrosis-associated genes in lung tissues. See Mediators of Inflammation, vol. 2018, Article ID 7967868, “Serotonin Exhibits Accelerated Bleomycin-Induced Pulmonary Fibrosis through TPH1 Knockout Mouse Experiments”. In another bleomycin-induced mouse model, the efficacy of 5-HT7 receptor antagonist SB-269970 in attenuating pulmonary fibrosis was compared to that of 5-HT2A/B receptor antagonist terguride. Both receptor antagonists were attenuating in both the inflammatory phase and the fibrogenic phase.

Without intending to be bound to any theory, it is believed the rate-limiting step in 5-HT biosynthesis is the hydroxylation of tryptophan by dioxygen, which is catalyzed by tryptophan hydroxylase (TPH; EC 1.14.16.4) in the presence of the cofactor (6R)-L-erythro-5,6,7,8-tetrahydrobiopterin (BH4). The resulting oxidized product, 5-hydroxy tryptophan (5-HTT) is subsequently decarboxylated by an aromatic amino acid decarboxylase (AAAD; EC 4.1.1.28) to produce 5-HT. Together with phenylalanine hydroxylase (PheOH) and tyrosine hydroxylase (TH), TPH belongs to the pterin-dependent aromatic amino acid hydroxylase family.

Two vertebrate isoforms of TPH, namely TPH1 and TPH2, have been identified. TPH1 is primarily expressed in the pineal gland and non-neuronal tissues, such as enterochromaffin (EC) cells located in the gastrointestinal (GI) tract. TPH2 (the dominant form in the brain) is expressed exclusively in neuronal cells, such as dorsal raphe or myenteric plexus cells. The peripheral and central systems involved in 5-HT biosynthesis are isolated, with 5-HT being unable to cross the blood-brain barrier. Therefore, the pharmacological effects of 5-HT can be modulated by agents affecting TPH in the periphery, mainly TPH1 in the gut.

WO 2015/035113 and U.S. Pat. No. 9,199,994 disclose spirocyclic compounds that act as inhibitors of THP and are useful in the treatment of various diseases and disorders associated with peripheral serotonin, including cardiovascular diseases of pulmonary arterial hypertension (PAH) and associated pulmonary arterial hypertension (APAH).

There is a need for a method of treating or preventing ILD, including IPF and PF in a patient. There is a need for a method of treating or preventing ILD, including IPF and PF in a patient with a pharmaceutical compound or composition. There is yet further a need for a method of treating or preventing ILD, including IPF and PF in a patient with a pharmaceutical compound or composition containing a THP1 inhibitor.

### SUMMARY OF THE DISCLOSURE

According to the present disclosure, there is provided a method of treating or preventing interstitial lung disease (ILD) in a patient. The method includes the step of administering to the patient a therapeutically effective amount of a THP1 inhibitor.

Further according to the present disclosure, there is provided method of treating or preventing pulmonary fibrosis (PF) in a patient. The method includes the step of administering to the patient a therapeutically effective amount of a THP1 inhibitor.

Further according to the present disclosure, there is provided a method of treating or preventing interstitial pulmonary fibrosis (IPF) in a patient. The method includes the step of administering to the patient a therapeutically effective amount of a THP1 inhibitor.

Further according to the present disclosure, there is provided a method for treating or preventing ILD, including IPF, in a patient. The method has the step of administering a therapeutically effective amount of a THP1 inhibitor in the form of a spirocyclic compound of the following formula:

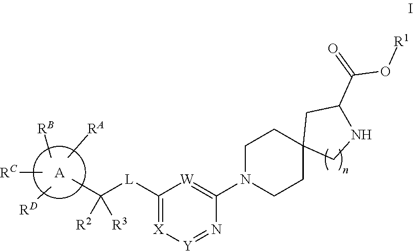

or a pharmaceutically acceptable salt thereof, wherein:

Ring A is C₃₋₁₀ cycloalkyl, C₆₋₁₀ aryl, 4 to 10-membered heterocycloalkyl, or 5 to 10-membered heteroaryl;

L is O or NR⁴;

W is N or CR⁵;

X is N or CR⁶;

Y is N or CR⁷;

wherein only one of X and Y is N;

R¹ is H, C₁₋₁₀ alkyl, C₃₋₁₀ cycloalkyl, phenyl, —(CR⁸R⁹)ₚOC(O)R¹⁰, —(CR⁸R⁹)ₚNR¹¹R¹², or —(CR⁸R⁹)ₚC(O)NR¹¹R¹², wherein said C₁₋₁₀ alkyl, C₃₋₁₀ cycloalkyl, and phenyl are each optionally substituted with 1, 2, 3, 4, or 5 substituents independently selected from F, Cl, Br, CN, C₁₋₄alkyl, and C₁₋₄ haloalkyl;

R² and R³ are each independently selected from H, C₁₋₄alkyl, and C₁₋₄ haloalkyl;

R⁴ is H or C₁₋₄alkyl;

R⁵ and R⁶ are each independently selected from H, halo, and C₁₋₄alkyl;

R⁷ is H, C₁₋₄alkyl, C₂₋₆ alkenyl, C₃₋₁₀ cycloalkyl, C₃₋₁₀ cycloalkyl-C₁₋₄ alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, (5-10 membered heteroaryl)-C₁₋₄alkyl, NR¹³R¹⁴, OR¹⁵, C(O)R¹⁶, S(O)_{q}R¹⁷, wherein said C₁₋₄alkyl, C₂₋₆ alkenyl, C₃₋₁₀ cycloalkyl, C₃₋₁₀ cycloalkyl-C₁₋₄ alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, and (5-10 membered heteroaryl)-C₁₋₄alkyl are each optionally substituted by 1, 2, or 3 substituents selected from halo, C₁₋₄alkyl, C₂₋₆ alkenyl, amino, C₁₋₄ alkylamino, C₂₋₈ dialkylamino, hydroxy, and C₁₋₄alkoxy;

R⁸ and R⁹ are each independently selected from H and C₁₋₄alkyl;

R¹⁰ is C₁₋₆alkyl optionally substituted by 1, 2 or 3 substituents independently selected from C₁₋₆ haloalkyl, C₃₋₁₀ cycloalkyl, OR^{a}, and NR^{c}R^{d};

R¹¹ and R¹² are each independently selected from H and C₁₋₆alkyl;

R¹³ is H or C₁₋₄alkyl;

R¹⁴ is H, C₁₋₄alkyl, C₃₋₇ cycloalkyl, C₃₋₇cycloalkyl-C₁₋₄alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, or (5-10 membered heteroaryl)-C₁₋₄alkyl, C(O)R^{b1}, C(O)OR^{a1}, C(O)NR^{c1}R^{d1}, S(O)R^{b1}, S(O)₂R^{b1}, or S(O)₂NR^{c1}R^{d1}, wherein said C₁₋₄alkyl, C₃₋₇ cycloalkyl, C₃₋₇cycloalkyl-C₁₋₄alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, and (5-10 membered heteroaryl)-C₁₋₄alkyl are each optionally substituted by 1, 2, or 3 substituents independently selected from halo, C₁₋₄ alkyl, C₁₋₄ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

or R¹³ and R¹⁴ together with the N atom to which they are attached form a 4-, 5-, 6-, or 7-membered heterocycloalkyl group optionally substituted with 1, 2, or 3 substituents independently selected from C₁₋₆ alkyl, C₃₋₇cycloalkyl, 4-7 membered heterocycloalkyl, C₆₋₁₀ aryl, 5-6 membered heteroaryl, halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};

R¹⁵ is H, C₁₋₄ alkyl, C₃₋₇ cycloalkyl, C₃₋₇ cycloalkyl-C₁₋₄ alkyl, C₆₋₁₀ aryl, C₆₋₁₀ aryl-C₁₋₄ alkyl, 4-10 membered heterocycloalkyl, (4-10 membered heterocycloalkyl)-C₁₋₄alkyl, 5-10 membered heteroaryl, or (5-10 membered heteroaryl)-C₁₋₄alkyl, wherein said C₁₋₄alkyl, C₃₋₇ cycloalkyl, C₃₋₇ cycloalkyl-C₁₋₄ alkyl, C₆₋₁₀ aryl, C₆₋₁₀ aryl-C₁₋₄ alkyl, 4-10 membered heterocycloalkyl, (4-10 membered heterocycloalkyl)-C₁₋₄alkyl, 5-10 membered heteroaryl, and (5-10 membered heteroaryl)-C₁₋₄alkyl are each optionally substituted by 1, 2, or 3 substituents independently selected from halo, C₃₋₇ cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};

R¹⁶ is C₁₋₄ alkyl or NR^{18a}R^{18b} wherein said C₁₋₄alkyl is optionally substituted by 1, 2, or 3 substituents independently selected from halo, C₃₋₇cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};

R¹⁷ is C₁₋₄ alkyl, NR^{18a}R^{18b}, or OR^{18c}, wherein said C₁₋₄alkyl is optionally substituted by 1, 2, or 3 substituents independently selected from halo, C₃₋₇ cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};

R^{18a} and R^{18b} are each independently selected from H and C₁₋₄ alkyl wherein said C₁₋₄ alkyl is optionally substituted by 1, 2, or 3 substituents independently selected from halo, C₃₋₇cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c4}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1}; or

R^{18a} and R^{18b} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};

R^{18c} is H, C₁₋₆ alkyl, C₃₋₁₀ cycloalkyl, C₃₋₇cycloalkyl-C₁₋₄alkyl, C₆₋₁₀ aryl, C₆₋₁₀ aryl-C₁₋₄ alkyl, 4-10 membered heterocycloalkyl, (4-10 membered heterocycloalkyl)-C₁₋₄alkyl, 5-10 membered heteroaryl, or (5-10 membered heteroaryl)-C₁₋₄alkyl, wherein said C₁₋₆alkyl, C₃₋₇ cycloalkyl, C₃₋₁₀ cycloalkyl-C₁₋₄alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, and (5-10 membered heteroaryl)-C₁₋₄alkyl are each optionally substituted by 1, 2, or 3 substituents independently selected from halo, C₁₋₄ alkyl, C₁₋₄ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R^{A} is H, Cy¹, halo, C₁₋₆ alkyl, C₂₋₆ alkenyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, or S(O)₂NR^{c2}R^{d2}, wherein said C₁₋₆ alkyl and C₂₋₆ alkenyl are each optionally substituted with 1, 2, 3, 4, or 5 substituents independently selected from Cy¹, halo, C₁₋₆ alkyl, C₂₋₆ alkenyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

R^{B} is H, Cy², halo, C₁₋₆ alkyl, C₂₋₆ alkenyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, or S(O)₂NR^{c3}R^{d3}, wherein said C₁₋₆ alkyl and C₂₋₆ alkenyl are each optionally substituted with 1, 2, 3, 4, or 5 substituents independently selected from Cy², halo, C₁₋₆ alkyl, C₂₋₆ alkenyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c1}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};

R^{C} and R^{D} are independently selected from H, halo, C₁₋₆ alkyl, C₂₋₆ alkenyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl and C₂₋₆ alkenyl are each optionally substituted with 1, 2, 3, 4, or 5 substituents independently selected from C₆₋₁₀ aryl, C₃₋₁₀ cycloalkyl, 5-10 membered heteroaryl, 4-10 membered heterocycloalkyl, halo, C₁₋₆ alkyl, C₂₋₆ alkenyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};

Cy¹ and Cy² are each independently selected from C₆₋₁₀ aryl, C₃₋₁₀ cycloalkyl, 5-10 membered heteroaryl, and 4-10 membered heterocycloalkyl, each of which is optionally substituted by 1, 2, 3, 4, or 5 substituents independently selected from R^{Cy};

each R^{Cy} is independently selected from halo, C₁₋₆ alkyl, C₁₋₆ haloalkyl, C₂₋₆ alkenyl, C₆₋₁₀ aryl, C₃₋₁₀ cycloalkyl, 5-10 membered heteroaryl, 4-10 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₂₋₆ alkenyl C₆₋₁₀ aryl, C₃₋₁₀ cycloalkyl, 5-10 membered heteroaryl, and 4-10 membered heterocycloalkyl are each optionally substituted with 1, 2, 3, 4, or 5 substituents independently selected from halo, C₁₋₆ alkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};

each R^{a}, R^{a1}, R^{a2}, R^{a3}, R^{a4}, and R^{a5} is independently selected from H, C₁₋₆ alkyl, C₁₋₄ haloalkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, or (4-10 membered heterocycloalkyl)-C₁₋₄alkyl, wherein said C₁₋₆ alkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄alkyl, and (4-10 membered heterocycloalkyl)-C₁₋₄alkyl are each optionally substituted with 1, 2, 3, 4, or 5 substituents independently selected from C₁₋₄ alkyl, halo, CN, OR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each R^{b1}, R^{b2}, R^{b3}, R^{b4}, and R^{b5} is independently selected from H, C₁₋₆ alkyl, C₁₋₄ haloalkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, or (4-10 membered heterocycloalkyl)-C₁₋₄alkyl, wherein said C₁₋₆ alkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄alkyl, and (4-10 membered heterocycloalkyl)-C₁₋₄alkyl are each optionally substituted with 1, 2, 3, 4, or 5 substituents independently selected from C₁₋₄ alkyl, halo, CN, OR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each R^{c}, R^{d}, R^{c1}, R^{d1}, R^{c2}, R^{d2}, R^{c3}, R^{d3}, R^{c4}, R^{d4}, R^{c5}, and R^{d5} is independently selected from H, C₁₋₆ alkyl, C₁₋₄ haloalkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄alkyl, C₃₋₁₀ cycloalkyl-C₁₋₄alkyl, (5-10 membered heteroaryl)-C₁₋₄alkyl, or (4-10 membered heterocycloalkyl)-C₁₋₄alkyl, wherein said C₁₋₆ alkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄alkyl are each optionally substituted with 1, 2, 3, 4, or 5 substituents independently selected from C₁₋₄ alkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c} and R^{d} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c1} and R^{d1} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c2} and R^{d2} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, and 5-6 membered heteroaryl, C₁₋₆ haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c3} and R^{d3} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, C₁₋₆haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c4} and R^{d4} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, C₁₋₆ haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c5} and R^{d5} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, C₁₋₆haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each R^{a6}, R^{b6}, R^{c6}, and R^{d6} is independently selected from H, C₁₋₄ alkyl, C₂₋₄ alkenyl, C₃₋₇ cycloalkyl, phenyl, 5-6 membered heteroaryl, and 4-7 membered heterocycloalkyl, wherein said C₁₋₄ alkyl, C₂₋₄ alkenyl, C₃₋₇ cycloalkyl, phenyl, 5-6 membered heteroaryl, and 4-7 membered heterocycloalkyl are each optionally substituted by 1, 2, or 3 substituents independently selected from OH, CN, amino, halo, C₁₋₄alkyl, C₁₋₄alkoxy, C₁₋₄alkylthio, C₁₋₄alkylamino, and di(C₁₋₄alkyl)amino;

n is 1 or 2;

p is 1, 2, or 3; and

q is 1 or 2;

wherein any aforementioned 4-10 or 4-7 membered heterocycloalkyl group optionally comprises 1, 2, or 3 oxo substituents, wherein each oxo substituent that is present is substituted on a ring-forming carbon, nitrogen, or sulfur atom of the 4-10 or 4-7 membered heterocycloalkyl group.

Further according to the present disclosure, there is provided a pharmaceutical composition for treating or preventing interstitial lung disease in a patient. The method includes the step of administering a therapeutically effective amount of a THP1 inhibitor in the form of the spirocyclic compound described above. The composition has the spirocyclic compound and a pharmaceutically acceptable excipient.

### BRIEF DESCRIPTION OF THE DRAWINGS

Embodiments of the present disclosure are described herein with reference to the following figures.

FIG. 1 is a plot of an XRPD of a crystalline compound of (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate according to the present disclosure (crystalline Form 3).

FIG. 2 is a plot of an XRPD of a crystalline compound of (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate of a different polymorphic form than that of FIG. 1 (crystalline Form 1).

### DETAILED DESCRIPTION OF THE DISCLOSURE

THP1 inhibitors useful in the present disclosure include the spirocyclic compounds disclosed herein.

The spirocyclic compound useful in inhibiting tryptophan hydroxylase is the following:

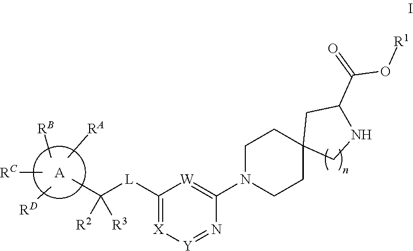

or a pharmaceutically acceptable salt thereof, wherein:

Ring A is C₃₋₁₀ cycloalkyl, C₆₋₁₀ aryl, 4 to 10-membered heterocycloalkyl, or 5 to 10-membered heteroaryl;

L is O or NR⁴;

W is N or CR⁵;

X is N or CR⁶;

Y is N or CR⁷;

wherein only one of X and Y is N;

R¹ is H, C₁₋₁₀ alkyl, C₃₋₁₀ cycloalkyl, phenyl, —(CR⁸R⁹)ₚOC(O)R¹⁰, —(CR⁸R⁹)ₚNR¹¹R¹², or —(CR⁸R⁹)ₚC(O)NR¹¹R¹², wherein said C₁₋₁₀ alkyl, C₃₋₁₀ cycloalkyl, and phenyl are each optionally substituted with 1, 2, 3, 4, or 5 substituents independently selected from F, Cl, Br, CN, C₁₋₄alkyl, and C₁₋₄ haloalkyl;

R² and R³ are each independently selected from H, C₁₋₄alkyl, and C₁₋₄ haloalkyl;

R⁴ is H or C₁₋₄alkyl;

R⁵ and R⁶ are each independently selected from H, halo, and C₁₋₄alkyl;

R⁷ is H, C₁₋₄alkyl, C₂₋₆ alkenyl, C₃₋₁₀ cycloalkyl, C₃₋₁₀ cycloalkyl-C₁₋₄ alkyl, C₆₋₁₀ aryl, C₆₋₁₀ aryl-C₁₋₄alkyl, 4-10 membered heterocycloalkyl, (4-10 membered heterocycloalkyl)-C₁₋₄ alkyl, 5-10 membered heteroaryl, (5-10 membered heteroaryl)-C₁₋₄alkyl, NR¹³R¹⁴, OR¹⁵, C(O)R¹⁶, S(O)_{q}R¹⁷, wherein said C₁₋₄alkyl, C₂₋₆ alkenyl, C₃₋₁₀ cycloalkyl, C₃₋₁₀ cycloalkyl-C₁₋₄ alkyl, C₆₋₁₀ aryl, C₆₋₁₀ aryl-C₁₋₄alkyl, 4-10 membered heterocycloalkyl, (4-10 membered heterocycloalkyl)-C₁₋₄ alkyl, 5-10 membered heteroaryl, and (5-10 membered heteroaryl)-C₁₋₄alkyl are each optionally substituted by 1, 2, or 3 substituents selected from halo, C₁₋₄alkyl, C₂₋₆ alkenyl, amino, C₁₋₄ alkylamino, C₂₋₈ dialkylamino, hydroxy, and C₁₋₄alkoxy;

R⁸ and R⁹ are each independently selected from H and C₁₋₄alkyl;

R¹⁰ is C₁₋₆alkyl optionally substituted by 1, 2 or 3 substituents independently selected from C₁₋₆ haloalkyl, C₃₋₁₀ cycloalkyl, OR^{a}, and NR^{c}R^{d};

R¹¹ and R¹² are each independently selected from H and C₁₋₆alkyl;

R¹³ is H or C₁₋₄alkyl;

R¹⁴ is H, C₁₋₄ alkyl, C₃₋₇ cycloalkyl, C₃₋₇ cycloalkyl-C₁₋₄ alkyl, C₆₋₁₀ aryl, C₆₋₁₀ aryl-C₁₋₄ alkyl, 4-10 membered heterocycloalkyl, (4-10 membered heterocycloalkyl)-C₁₋₄alkyl, 5-10 membered heteroaryl, or (5-10 membered heteroaryl)-C₁₋₄alkyl, C(O)R^{b1}, C(O)OR^{a1}, C(O)NR^{c1}R^{d1}, S(O)R^{b1}, S(O)₂R^{b1}, or S(O)₂NR^{c1}R^{d1}, wherein said C₁₋₄alkyl, C₃₋₇cycloalkyl, C₃₋₇cycloalkyl-C₁₋₄alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, and (5-10 membered heteroaryl)-C₁₋₄ alkyl are each optionally substituted by 1, 2, or 3 substituents independently selected from halo, C₁₋₄ alkyl, C₁₋₄ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; or

R¹³ and R¹⁴ together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};

R¹⁵ is H, C₁₋₄ alkyl, C₃₋₇ cycloalkyl, C₃₋₇ cycloalkyl-C₁₋₄ alkyl, C₆₋₁₀ aryl, C₆₋₁₀ aryl-C₁₋₄ alkyl, 4-10 membered heterocycloalkyl, (4-10 membered heterocycloalkyl)-C₁₋₄alkyl, 5-10 membered heteroaryl, or (5-10 membered heteroaryl)-C₁₋₄alkyl, wherein said C₁₋₄alkyl, C₃₋₇ cycloalkyl, C₃₋₇ cycloalkyl-C₁₋₄ alkyl, C₆₋₁₀ aryl, C₆₋₁₀ aryl-C₁₋₄ alkyl, 4-10 membered heterocycloalkyl, (4-10 membered heterocycloalkyl)-C₁₋₄alkyl, 5-10 membered heteroaryl, and (5-10 membered heteroaryl)-C₁₋₄alkyl are each optionally substituted by 1, 2, or 3 substituents independently selected from halo, C₃₋₇ cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};

R¹⁶ is C₁₋₄ alkyl or NR^{18a}R^{18b} wherein said C₁₋₄alkyl is optionally substituted by 1, 2, or 3 substituents independently selected from halo, C₃₋₇cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};

R¹⁷ is C₁₋₄ alkyl, NR^{18a}R^{18b}, or OR^{18c}, wherein said C₁₋₄alkyl is optionally substituted by 1, 2, or 3 substituents independently selected from halo, C₃₋₇ cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};

R^{18a} and R^{18b} are each independently selected from H and C₁₋₄ alkyl wherein said C₁₋₄ alkyl is optionally substituted by 1, 2, or 3 substituents independently selected from halo, C₃₋₇cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c4}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1}; or

R^{18a} and R^{18b} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};

R^{18c} is H, C₁₋₆ alkyl, C₃₋₁₀ cycloalkyl, C₃₋₇cycloalkyl-C₁₋₄alkyl, C₆₋₁₀ aryl, C₆₋₁₀ aryl-C₁₋₄ alkyl, 4-10 membered heterocycloalkyl, (4-10 membered heterocycloalkyl)-C₁₋₄alkyl, 5-10 membered heteroaryl, or (5-10 membered heteroaryl)-C₁₋₄alkyl, wherein said C₁₋₆alkyl, C₃₋₇ cycloalkyl, C₃₋₁₀ cycloalkyl-C₁₋₄alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, and (5-10 membered heteroaryl)-C₁₋₄alkyl are each optionally substituted by 1, 2, or 3 substituents independently selected from halo, C₁₋₄ alkyl, C₁₋₄ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R^{A} is H, Cy¹, halo, C₁₋₆ alkyl, C₂₋₆ alkenyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, or S(O)₂NR^{c2}R^{d2}, wherein said C₁₋₆ alkyl and C₂₋₆ alkenyl are each optionally substituted with 1, 2, 3, 4, or 5 substituents independently selected from Cy¹, halo, C₁₋₆ alkyl, C₂₋₆ alkenyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

R^{B} is H, Cy², halo, C₁₋₆ alkyl, C₂₋₆ alkenyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, or S(O)₂NR^{c3}R^{d3}, wherein said C₁₋₆ alkyl and C₂₋₆ alkenyl are each optionally substituted with 1, 2, 3, 4, or 5 substituents independently selected from Cy², halo, C₁₋₆ alkyl, C₂₋₆ alkenyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c1}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};

R^{C} and R^{D} are independently selected from H, halo, C₁₋₆ alkyl, C₂₋₆ alkenyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl and C₂₋₆ alkenyl are each optionally substituted with 1, 2, 3, 4, or 5 substituents independently selected from C₆₋₁₀ aryl, C₃₋₁₀ cycloalkyl, 5-10 membered heteroaryl, 4-10 membered heterocycloalkyl, halo, C₁₋₆ alkyl, C₂₋₆ alkenyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};

Cy¹ and Cy² are each independently selected from C₆₋₁₀ aryl, C₃₋₁₀ cycloalkyl, 5-10 membered heteroaryl, and 4-10 membered heterocycloalkyl, each of which is optionally substituted by 1, 2, 3, 4, or 5 substituents independently selected from R^{Cy};

each R^{Cy} is independently selected from halo, C₁₋₆ alkyl, C₁₋₆ haloalkyl, C₂₋₆ alkenyl, C₆₋₁₀ aryl, C₃₋₁₀ cycloalkyl, 5-10 membered heteroaryl, 4-10 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₂₋₆ alkenyl C₆₋₁₀ aryl, C₃₋₁₀ cycloalkyl, 5-10 membered heteroaryl, and 4-10 membered heterocycloalkyl are each optionally substituted with 1, 2, 3, 4, or 5 substituents independently selected from halo, C₁₋₆ alkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};

each R^{a}, R^{a1}, R^{a2}, R^{a3}, R^{a4}, and R^{a5} is independently selected from H, C₁₋₆ alkyl, C₁₋₄ haloalkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, or (4-10 membered heterocycloalkyl)-C₁₋₄alkyl, wherein said C₁₋₆ alkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄alkyl, and (4-10 membered heterocycloalkyl)-C₁₋₄alkyl are each optionally substituted with 1, 2, 3, 4, or 5 substituents independently selected from C₁₋₄ alkyl, halo, CN, OR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each R^{b1}, R^{b2}, R^{b3}, R^{b4}, and R^{b5} is independently selected from H, C₁₋₆ alkyl, C₁₋₄ haloalkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, or (4-10 membered heterocycloalkyl)-C₁₋₄alkyl, wherein said C₁₋₆ alkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄alkyl, and (4-10 membered heterocycloalkyl)-C₁₋₄alkyl are each optionally substituted with 1, 2, 3, 4, or 5 substituents independently selected from C₁₋₄ alkyl, halo, CN, OR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each R^{c}, R^{d}, R^{c1}, R^{d1}, R^{c2}, R^{d2}, R^{c3}, R^{d3}, R^{c4}, R^{d4}, R^{c5}, and R^{d5} is independently selected from H, C₁₋₆ alkyl, C₁₋₄ haloalkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄alkyl, C₃₋₁₀ cycloalkyl-C₁₋₄alkyl, (5-10 membered heteroaryl)-C₁₋₄alkyl, or (4-10 membered heterocycloalkyl)-C₁₋₄alkyl, wherein said C₁₋₆ alkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄alkyl are each optionally substituted with 1, 2, 3, 4, or 5 substituents independently selected from C₁₋₄ alkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c} and R^{d} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c1} and R^{d1} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c2} and R^{d2} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, and 5-6 membered heteroaryl, C₁₋₆ haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c3} and R^{d3} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, C₁₋₆ haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c4} and R^{d4} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, C₁₋₆haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or

any R^{c5} and R^{d5} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, C₁₋₆ haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each R^{a6}, R^{b6}, R^{c6}, and R^{d6} is independently selected from H, C₁₋₄ alkyl, C₂₋₄ alkenyl, C₃₋₇ cycloalkyl, phenyl, 5-6 membered heteroaryl, and 4-7 membered heterocycloalkyl, wherein said C₁₋₄ alkyl, C₂₋₄ alkenyl, C₃₋₇ cycloalkyl, phenyl, 5-6 membered heteroaryl, and 4-7 membered heterocycloalkyl are each optionally substituted by 1, 2, or 3 substituents independently selected from OH, CN, amino, halo, C₁₋₄alkyl, C₁₋₄alkoxy, C₁₋₄alkylthio, C₁₋₄alkylamino, and di(C₁₋₄alkyl)amino;

n is 1 or 2;

p is 1, 2, or 3; and

q is 1 or 2;

wherein any aforementioned 4-10 or 4-7 membered heterocycloalkyl group optionally comprises 1, 2, or 3 oxo substituents, wherein each oxo substituent that is present is substituted on a ring-forming carbon, nitrogen, or sulfur atom of the 4-10 or 4-7 membered heterocycloalkyl group.

Preferred embodiments of compounds corresponding to Formula I include the following: wherein L is O; wherein L is NR⁴; wherein W is CR⁵, X is N, and Y is CR⁷; wherein W is N, X is N, and Y is CR⁷; wherein W is CR⁵, X is CR⁶, and Y is N; wherein W is CR⁵, X is CR⁶, and Y is CR⁷; wherein W is N, X is CR⁶, and Y is CR⁷; wherein R² is H and R³ is H; wherein R² is H and R³ is C₁₋₄alkyl; wherein R² is H and R³ is methyl; wherein R² is H and R³ is C₁₋₄ haloalkyl; wherein for the compound or a pharmaceutically acceptable salt thereof, the R² is H and R³ is trifluoromethyl; wherein n is 1; n is 2; R¹ is H; wherein R¹ is C₁₋₁₀ alkyl, C₃₋₁₀ cycloalkyl, phenyl, —(CR⁸R⁹)ₚOC(O)R¹⁰, —(CR⁸R⁹)ₚNR¹¹R¹², or —(CR⁸R⁹)ₚC(O)NR¹¹R¹², wherein said C₁₋₁₀ alkyl, C₃₋₁₀ cycloalkyl, and phenyl are each optionally substituted with 1, 2, 3, 4, or 5 substituents independently selected from F, Cl, Br, CN, C₁₋₄alkyl, and C₁₋₄ haloalkyl; wherein R¹ is C₁₋₁₀ alkyl; R⁴ is H; wherein R⁵ is H; R⁶ is H; wherein R⁷ is other than H; wherein R⁷ is C₁₋₄alkyl, NR¹³R¹⁴, or OR¹⁵; wherein R⁷ is NR¹³R¹⁴; wherein R⁷ is NH₂; R⁷ is C₁₋₄alkyl; wherein R⁷ is OR¹⁵; wherein Ring A is C₃₋₁₀ cycloalkyl; wherein Ring A is C₆₋₁₀ aryl; Ring A is phenyl; wherein Ring A is 4 to 10-membered heterocycloalkyl; wherein Ring A is phenyl, adamantanyl, naphthyl, 1,2,3,4-tetrahydroquinoxalinyl, 3,4-dihydroqinazolinyl, 1,2,3,4-tetrahydroquinazolinyl, or pyridyl; wherein Ring A is 5 to 10-membered heteroaryl; wherein at least one of R^{A}, R^{B}, R^{c}, and R^{D} is other than hydrogen; wherein at least two of R^{A}, R^{B}, R^{c}, and R^{D} are other than hydrogen; wherein R^{A} is Cy¹; wherein R^{A} is C₆₋₁₀ aryl or 5-10 membered heteroaryl, each of which is optionally substituted by 1, 2, 3, 4, or 5 substituents independently selected from R^{Cy}; wherein R^{A} is 5-10 membered heteroaryl which is optionally substituted by 1, 2, 3, 4, or 5 substituents independently selected from R^{Cy}; wherein R^{A} is pyrazolyl which is optionally substituted by 1, 2, 3, 4, or 5 substituents independently selected from R^{Cy}; wherein R^{A} is 3-methyl-1H-pyrazol-1-yl; wherein R^{A} is C₆₋₁₀ aryl optionally substituted by 1, 2, 3, 4, or 5 substituents independently selected from R^{Cy}; wherein R^{A} is phenyl optionally substituted by 1, 2, or 3 substituents independently selected from R^{Cy}; wherein R^{B} is H; wherein R^{B} is Cy², halo, C₁₋₆ alkyl, C₂₋₆ alkenyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, or S(O)₂NR^{c3}R^{d3}, wherein said C₁₋₆ alkyl and C₂₋₆ alkenyl are each optionally substituted with 1, 2, 3, 4, or 5 substituents independently selected from Cy², halo, C₁₋₆ alkyl, C₂₋₆ alkenyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c1}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein R^{B} is Cy²; wherein R^{B} is C₆₋₁₀ aryl or 5-10 membered heteroaryl, each of which is optionally substituted by 1, 2, 3, 4, or 5 substituents independently selected from R^{Cy}; wherein R^{B} is halo, C₁₋₆ alkyl, C₂₋₆ alkenyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, or S(O)₂NR^{c3}R^{d3}, wherein said C₁₋₆ alkyl and C₂₋₆ alkenyl are each optionally substituted with 1, 2, 3, 4, or 5 substituents independently selected from Cy², halo, C₁₋₆ alkyl, C₂₋₆ alkenyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c1}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein R^{B} is halo; wherein R^{C} is H; wherein R^{c} is halo, C₁₋₆ alkyl, C₂₋₆ alkenyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, or S(O)₂NR^{c4}R^{d4}; wherein said C₁₋₆ alkyl and C₂₋₆ alkenyl are each optionally substituted with 1, 2, 3, 4, or 5 substituents independently selected from C₆₋₁₀ aryl, C₃₋₁₀ cycloalkyl, 5-10 membered heteroaryl, 4-10 membered heterocycloalkyl, halo, C₁₋₆ alkyl, C₂₋₆ alkenyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein R^{D} is H; and wherein R^{D} is halo, C₁₋₆ alkyl, C₂₋₆ alkenyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, or S(O)₂NR^{c4}R^{d4}, and wherein said C₁₋₆ alkyl and C₂₋₆ alkenyl are each optionally substituted with 1, 2, 3, 4, or 5 substituents independently selected from C₆₋₁₀ aryl, C₃₋₁₀ cycloalkyl, 5-10 membered heteroaryl, 4-10 membered heterocycloalkyl, halo, C₁₋₆ alkyl, C₂₋₆ alkenyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}.

Useful spirocyclic compounds can be represented by the following formulas:

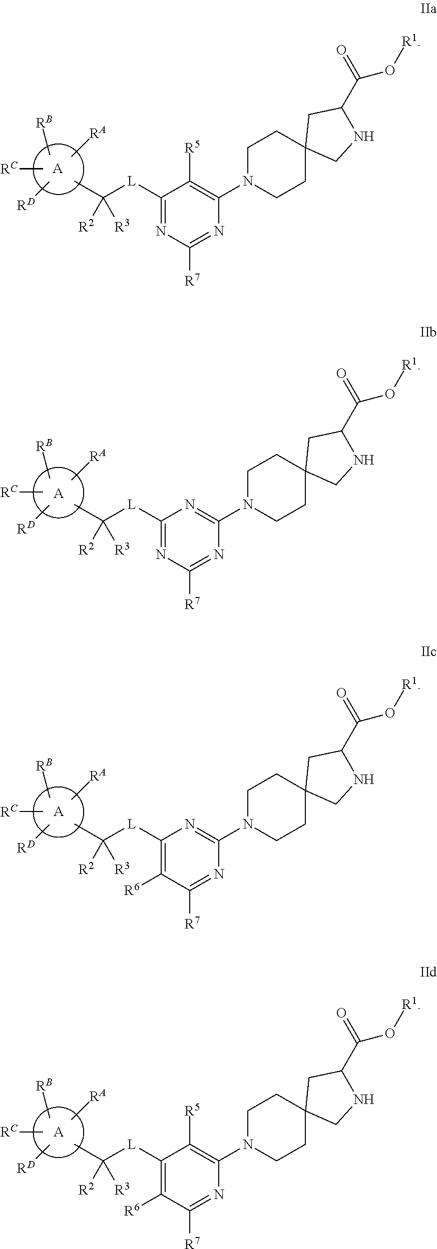

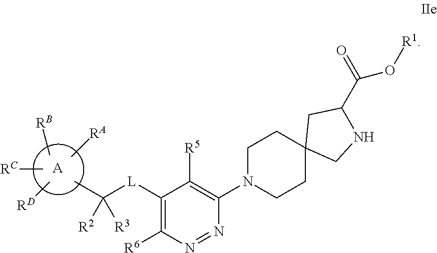

Preferred embodiments of compounds corresponding to Formulas IIa to IIe include the following: wherein L is O; wherein L is NR₄; wherein R³ is H; R² is CF₃ and R³ is H; wherein R¹ is H or C₁₋₁₀alkyl; wherein R^{A} is 5-10 membered heteroaryl which is optionally substituted by 1, 2, 3, 4, or 5 substituents independently selected from R^{Cy}; wherein R^{A} is 5 to 6-membered heteroaryl optionally substituted by 1, 2, or 3 substituents independently selected from R^{Cy}; wherein R^{A} is C₆₋₁₀ aryl optionally substituted by 1, 2, 3, 4, or 5 substituents independently selected from R^{Cy}; wherein R^{A} is phenyl optionally substituted by 1, 2, or 3 substituents independently selected from R^{Cy}; wherein R^{B} is Cy²; wherein R^{B} is H, halo, C₁₋₆ alkyl, C₂₋₆ alkenyl, C₁₋₆ haloalkyl, CN, OR^{a3}, C(O)NR^{c3}R^{d3}, or C(O)OR^{a3}, wherein said C₁₋₆ alkyl and C₂₋₆ alkenyl are each optionally substituted with 1, 2, or 3 substituents independently selected from halo, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c1}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein R^{C} is H; wherein R^{D} is H; wherein R⁵ is H; and R⁶ is H.

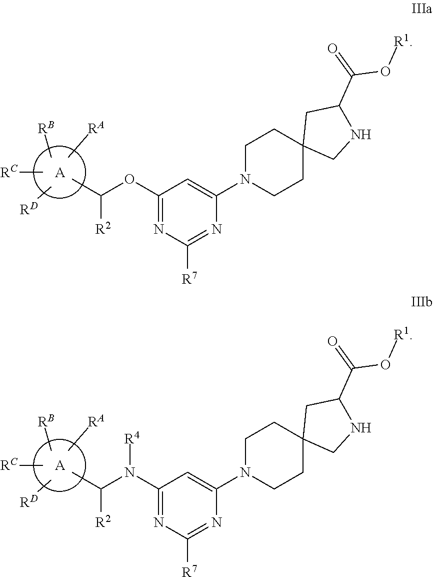

Preferred embodiments of compounds corresponding to Formulas IIIa and IIIb include the following: wherein R² is CF₃; R¹ is H or C₁₋₁₀alkyl; wherein R^{A} is 5-10 membered heteroaryl which is optionally substituted by 1, 2, 3, 4, or 5 substituents independently selected from R^{Cy}; wherein R^{A} is 5 to 6-membered heteroaryl optionally substituted by 1, 2, or 3 substituents independently selected from R^{Cy} or R^{A} is C₆₋₁₀ aryl optionally substituted by 1, 2, 3, 4, or 5 substituents independently selected from R^{Cy}; wherein R^{A} is phenyl optionally substituted by 1, 2, or 3 substituents independently selected from R^{Cy}; wherein R^{B} is Cy²; wherein R^{B} is H, halo, C₁₋₆ alkyl, C₂₋₆ alkenyl, C₁₋₆ haloalkyl, CN, OR^{a3}, C(O)NR^{c3}R^{d3}, or C(O)OR^{a3}, wherein said C₁₋₆ alkyl and C₂₋₆ alkenyl are each optionally substituted with 1, 2, or 3 substituents independently selected from halo, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c1}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; and wherein R^{C} is H; wherein R^{D} is H.

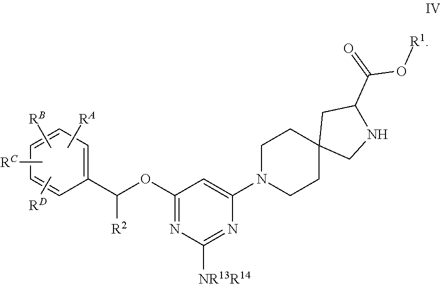

Preferred embodiments of compounds corresponding to Formula IV include the following: wherein R² is CF₃; R¹ is H or C₁₋₁₀alkyl; wherein R^{A} is 5-10 membered heteroaryl which is optionally substituted by 1, 2, 3, 4, or 5 substituents independently selected from R^{Cy}; wherein R^{A} is 5 to 6-membered heteroaryl optionally substituted by 1, 2, or 3 substituents independently selected from R^{Cy}; wherein R^{A} is C₆₋₁₀ aryl optionally substituted by 1, 2, 3, 4, or 5 substituents independently selected from R^{Cy}; wherein R^{A} is phenyl optionally substituted by 1, 2, or 3 substituents independently selected from R^{Cy}; wherein R^{B} is Cy²; wherein R^{B} is H, halo, C₁₋₆ alkyl, C₂₋₆ alkenyl, C₁₋₆ haloalkyl, CN, OR^{a3}, C(O)NR^{c3}R^{d3}, or C(O)OR^{a3}, wherein said C₁₋₆ alkyl and C₂₋₆ alkenyl are each optionally substituted with 1, 2, or 3 substituents independently selected from halo, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c1}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein for the compound or a pharmaceutically acceptable salt thereof, the R^{C} is H; and wherein R^{D} is H.

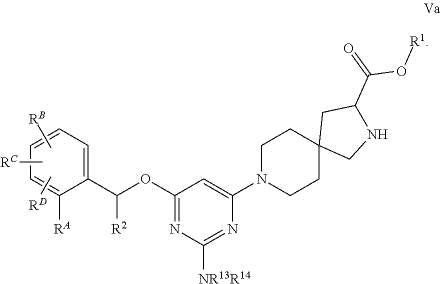

Preferred embodiments of compounds corresponding to Formula Va. include the following: wherein R² is CF₃; R¹ is H or C₁₋₁₀alkyl; wherein R^{A} is 5-10 membered heteroaryl optionally substituted by 1, 2, 3, 4, or 5 substituents independently selected from R^{Cy}; wherein R^{A} is 5 to 6-membered heteroaryl optionally substituted by 1, 2, or 3 substituents independently selected from R^{Cy}; wherein R^{A} is C₆₋₁₀ aryl optionally substituted by 1, 2, or 3 substituents independently selected from R^{Cy}; wherein the R^{A} is phenyl optionally substituted by 1, 2, or 3 substituents independently selected from R^{Cy}; wherein the R^{B} is Cy²; wherein R^{B} is H, halo, C₁₋₆ alkyl, C₂₋₆ alkenyl, C₁₋₆ haloalkyl, CN, OR^{a3}, C(O)NR^{c3}R^{d3}, or C(O)OR^{a3}, and wherein said C₁₋₆ alkyl and C₂₋₆ alkenyl are each optionally substituted with 1, 2, or 3 substituents independently selected from halo, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c1}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

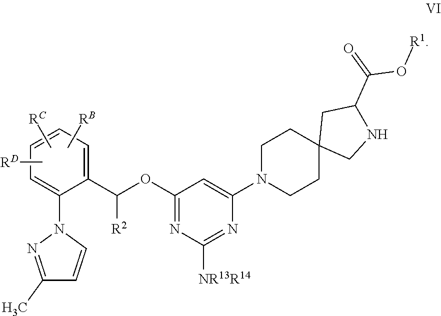

Preferred embodiments of compounds corresponding to Formula VI include the following: wherein R² is CF₃; wherein R¹ is H or C₁₋₁₀alkyl; wherein R^{B} is Cy²; wherein R^{B} is H, halo, C₁₋₆ alkyl, C₂₋₆ alkenyl, C₁₋₆ haloalkyl, CN, OR^{a3}, C(O)NR^{c3}R^{d3}, or C(O)OR^{a3}, wherein said C₁₋₆ alkyl and C₂₋₆ alkenyl are each optionally substituted with 1, 2, or 3 substituents independently selected from halo, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c1}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein R^{C} is H; and wherein R^{D} is H.

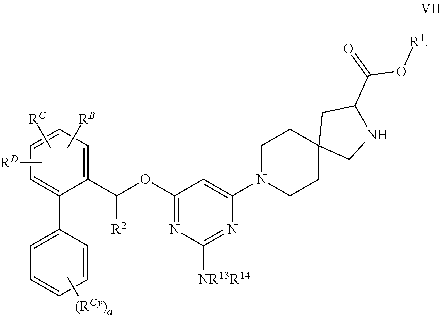

wherein a is 0, 1, 2, or 3.

Preferred embodiments of compounds corresponding to Formula VII include the following: wherein R² is CF₃; R¹ is H or C₁₋₁₀alkyl; wherein R^{B} is Cy²; wherein R^{B} is H, halo, C₁₋₆ alkyl, C₂₋₆ alkenyl, C₁₋₆ haloalkyl, CN, OR^{a3}, C(O)NR^{c3}R^{d3}, or C(O)OR^{a3}, wherein said C₁₋₆ alkyl and C₂₋₆ alkenyl are each optionally substituted with 1, 2, or 3 substituents independently selected from halo, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c1}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; R^{C} is H; R^{D} is H; wherein R^{Cy} is halo, C₁₋₆ alkyl, C₁₋₆ haloalkyl, 4-10 membered heterocycloalkyl, CN, NO₂, OR^{a5}, SR^{a5}, C(O)R^{b5}, C(O)NR^{c5}R^{d5}, C(O)OR^{a5}, NR^{c5}R^{d5}, S(O)₂R^{b5}, and S(O)₂NR^{c5}R^{d5}, wherein said C₁₋₆ alkyl and 4-10 membered heterocycloalkyl are each optionally substituted with 1, 2, 3, 4, or 5 substituents independently selected from halo, C₁₋₆ alkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein the chiral carbon to which —C(O)OR¹ is attached has an S configuration; and wherein the carbon to which —R² is attached has an R configuration.

A preferred spirocyclic compound is (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate (alternately referred to herein as “Compound A”). The compound has the formula

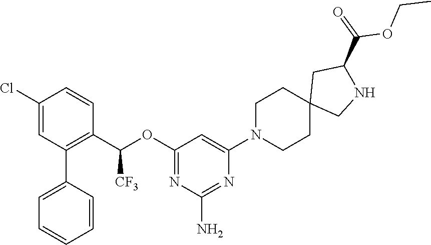 [SMILES: CCOC(=O)[C@@H]1CC2(CCN(c3cc(O[C@H](c4ccc(Cl)cc4-c4ccccc4)C(F)(F)F)nc(N)n3)CC2)CN1]

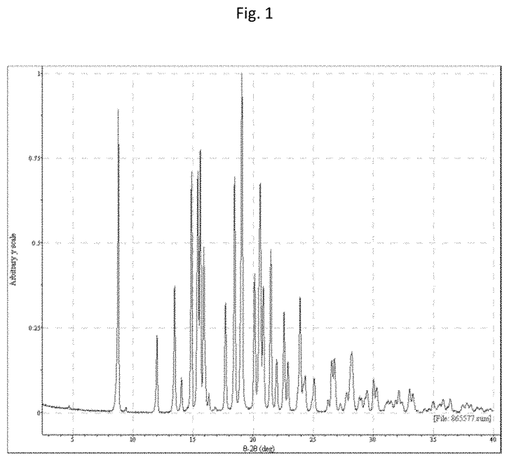

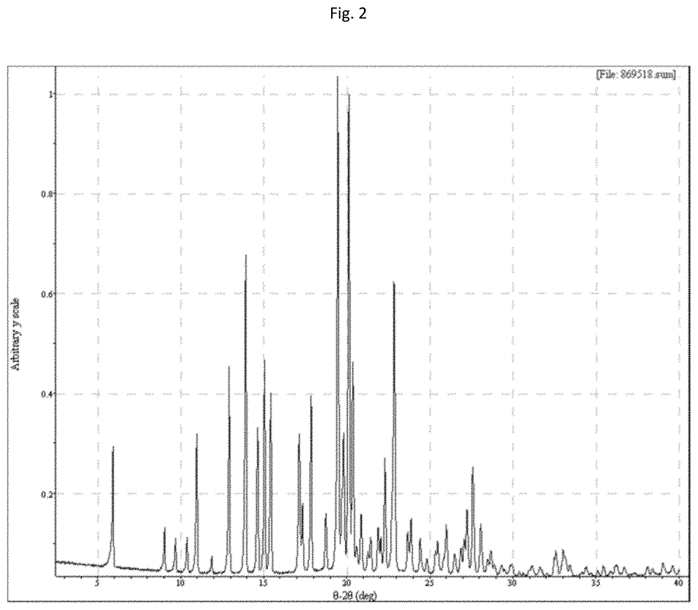

The compound may be used to prevent or treat IPF in either an amorphous or crystalline form. Two different polymorph crystalline forms are identified by x-ray powder diffraction patterns set forth in FIG. 1 (Form 3) and FIG. 2 (Form 1) as well as Tables 1 and 2 (Form 3) and Tables 3 and 4 (Form 1). The crystalline Form 3 polymorph is most preferred, as it exhibits substantially greater stability and shelf life compared to the crystalline Form 1 polymorph of the carboxylate compound, particularly at temperatures of less than 95° C.

TABLE 1
Observed Peaks for X-ray Powder Diffraction
Pattern for Compound A, Crystalline Form 3

| Peak position | | Intensity |
|---|---|---|
| (°2θ) | d space (Å) | (%) |
| 8.78 ± 0.20 | 10.077 ± 0.235 | 90 |
| 12.00 ± 0.20 | 7.375 ± 0.125 | 25 |
| 13.47 ± 0.20 | 6.573 ± 0.099 | 39 |
| 14.02 ± 0.20 | 6.316 ± 0.091 | 12 |
| 14.87 ± 0.20 | 5.956 ± 0.081 | 71 |
| 15.39 ± 0.20 | 5.757 ± 0.075 | 72 |
| 15.61 ± 0.20 | 5.677 ± 0.073 | 78 |
| 15.89 ± 0.20 | 5.576 ± 0.071 | 50 |
| 16.31 ± 0.20 | 5.434 ± 0.067 | 7 |
| 17.70 ± 0.20 | 5.011 ± 0.057 | 34 |
| 18.45 ± 0.20 | 4.809 ± 0.052 | 70 |
| 19.05 ± 0.20 | 4.658 ± 0.049 | 100 |
| 20.12 ± 0.20 | 4.413 ± 0.044 | 42 |
| 20.57 ± 0.20 | 4.317 ± 0.042 | 68 |
| 20.84 ± 0.20 | 4.262 ± 0.041 | 39 |
| 21.46 ± 0.20 | 4.141 ± 0.039 | 49 |
| 21.94 ± 0.20 | 4.051 ± 0.037 | 18 |
| 22.56 ± 0.20 | 3.941 ± 0.035 | 31 |
| 22.90 ± 0.20 | 3.884 ± 0.034 | 17 |
| 23.90 ± 0.20 | 3.723 ± 0.031 | 35 |
| 24.32 ± 0.20 | 3.660 ± 0.030 | 13 |
| 25.07 ± 0.20 | 3.552 ± 0.028 | 12 |
| 26.54 ± 0.20 | 3.359 ± 0.025 | 17 |
| 26.76 ± 0.20 | 3.332 ± 0.025 | 18 |
| 27.79 ± 0.20 | 3.210 ± 0.023 | 8 |
| 28.21 ± 0.20 | 3.163 ± 0.022 | 19 |
| 29.48 ± 0.20 | 3.030 ± 0.020 | 9 |

TABLE 2
Prominent Observed Peaks for X-ray Powder Diffraction
Pattern for Compound A, Crystalline Form 3

| Peak position | | Intensity |
|---|---|---|
| (°2θ) | d space (Å) | (%) |
| 8.78 ± 0.20 | 10.077 ± 0.235 | 90 |
| 14.87 ± 0.20 | 5.956 ± 0.081 | 71 |
| 15.39 ± 0.20 | 5.757 ± 0.075 | 72 |
| 15.61 ± 0.20 | 5.677 ± 0.073 | 78 |
| 18.45 ± 0.20 | 4.809 ± 0.052 | 70 |
| 19.05 ± 0.20 | 4.658 ± 0.049 | 100 |

In yet another aspect, the Form 3 crystalline polymorph exhibits a characteristic XRPD peak at 19.05±0.20 (° 2θ).

The Form 1 crystalline compound exhibits the XRPD (X-ray powder diffraction) pattern set forth below in Table 3.

TABLE 3
Observed Peaks for X-Ray Powder
Diffraction Pattern for Compound A,
Crystalline Form 1

| Peak position | d space | Intensity |
|---|---|---|
| (°2θ) | (Å) | (%) |
| 5.92 ± 0.20 | 14.936 ± 0.522 | 27 |
| 9.01 ± 0.20 | 9.816 ± 0.222 | 11 |
| 9.68 ± 0.20 | 9.140 ± 0.192 | 9 |
| 10.38 ± 0.20 | 8.523 ± 0.167 | 9 |
| 10.95 ± 0.20 | 8.082 ± 0.150 | 30 |
| 11.85 ± 0.20 | 7.468 ± 0.128 | 6 |
| 12.90 ± 0.20 | 6.861 ± 0.108 | 43 |
| 13.89 ± 0.20 | 6.376 ± 0.093 | 65 |
| 14.62 ± 0.20 | 6.057 ± 0.084 | 31 |
| 15.04 ± 0.20 | 5.890 ± 0.079 | 44 |
| 15.41 ± 0.20 | 5.750 ± 0.075 | 38 |
| 17.13 ± 0.20 | 5.176 ± 0.061 | 30 |
| 17.83 ± 0.20 | 4.974 ± 0.056 | 37 |
| 18.72 ± 0.20 | 4.741 ± 0.051 | 14 |
| 19.44 ± 0.20 | 4.567 ± 0.047 | 100 |
| 19.79 ± 0.20 | 4.487 ± 0.045 | 30 |
| 20.11 ± 0.20 | 4.417 ± 0.044 | 97 |
| 20.34 ± 0.20 | 4.366 ± 0.043 | 44 |
| 20.84 ± 0.20 | 4.262 ± 0.041 | 14 |
| 21.41 ± 0.20 | 4.151 ± 0.039 | 10 |
| 21.88 ± 0.20 | 4.063 ± 0.037 | 11 |
| 22.28 ± 0.20 | 3.991 ± 0.036 | 25 |
| 22.83 ± 0.20 | 3.895 ± 0.034 | 60 |
| 23.85 ± 0.20 | 3.731 ± 0.031 | 13 |
| 24.40 ± 0.20 | 3.648 ± 0.030 | 9 |
| 25.45 ± 0.20 | 3.500 ± 0.027 | 9 |
| 25.97 ± 0.20 | 3.431 ± 0.026 | 12 |
| 27.22 ± 0.20 | 3.276 ± 0.024 | 15 |
| 27.58 ± 0.20 | 3.235 ± 0.023 | 23 |
| 28.06 ± 0.20 | 3.180 ± 0.022 | 12 |
| 28.66 ± 0.20 | 3.115 ± 0.021 | 7 |

In still another aspect, the Form 1 crystalline compound exhibits prominent XRPD peaks set forth below in Table 4.

TABLE 4
Prominent Observed Peaks for X-Ray Powder
Diffraction Pattern for Compound A, Crystalline Form 1

| | | Intensity |
|---|---|---|
| Peak position (°2θ) | d space (Å) | (%) |
| 12.90 ± 0.20 | 6.861 ± 0.108 | 43 |
| 13.89 ± 0.20 | 6.376 ± 0.093 | 65 |
| 15.04 ± 0.20 | 5.890 ± 0.079 | 44 |
| 19.44 ± 0.20 | 4.567 ± 0.047 | 100 |
| 20.11 ± 0.20 | 4.417 ± 0.044 | 97 |
| 20.34 ± 0.20 | 4.366 ± 0.043 | 44 |
| 22.83 ± 0.20 | 3.895 ± 0.034 | 60 |

The amorphous form of the Compound A can be prepared by the method set forth in Example 63i of U.S. Pat. No. 9,199,994, wherein Example 63i is specifically incorporated herein by reference as well as U.S. Pat. No. 9,199,994 being incorporated herein in its entirety. The amorphous form can then be converted to crystalline form by extraction with organic solvents, such as C₄ to C₁₀ alcohols and ethers. Useful alcohols include pentane, hexane, and heptane. Useful ethers include methyl tert butyl ether (MTBE). By way of example, crystalline form 1 can be prepared by extraction with isopropanol, ethanol, cyclohexane, ethyl acetate, acetone, water, and mixtures of the foregoing, while crystalline form 3 can be prepared by extraction with MTBE and/or heptane. Crystalline forms 1 and 3 can be prepared by the extraction techniques set forth in U.S. Provisional Application No. 62/767,171, filed Nov. 14, 2018.

The efficacy of amorphous (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate in inhibiting TPH1 in mice was demonstrated in U.S. Pat. No. 9,199,994 in biological assays at Example 63i and Table 27.

Another preferred spirocyclic compound is (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid (alternately referred to herein as “Compound B”) of the formula

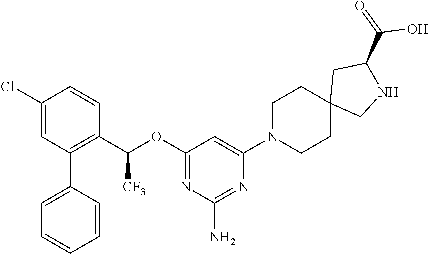 [SMILES: Nc1nc(O[C@H](c2ccc(Cl)cc2-c2ccccc2)C(F)(F)F)cc(N2CCC3(CC2)CN[C@H](C(=O)O)C3)n1]

The amorphous form of the Compound B can be prepared by the method set forth in Example 34c of U.S. Pat. No. 9,199,994.

The spirocyclic compounds are manufactured according to the processes described in the disclosure of U.S. Pat. No. 9,199,994, which is incorporated herein in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the enzyme with a compound of the disclosure includes the administration of a compound of the present disclosure to an individual or patient, such as a human, having the TPH1 enzyme, as well as, for example, introducing a compound of the disclosure into a sample containing a cellular or purified preparation containing the TPH1 enzyme.

As used herein, the term “individual” or “patient” used interchangeably, refers to any animal, including mammals, preferably mice, rats, other rodents, rabbits, dogs, cats, swine, cattle, sheep, horses, or primates, and, most preferably, humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” refers to inhibiting onset and/or worsening of the disease, and/or reduction of the risk onset, recurrence, or relapse; for example, in an individual who may be predisposed to the disease, condition or disorder but does not yet experience or display the pathology or symptomatology of the disease, or in an individual who has previously been treated for the disease, condition or disorder on one or more previous occasions and is asymptomatic for the pathology or symptomatology of the disease.

One or more additional pharmaceutical agents can be used in combination with the TPH1 inhibitor(s), such as the spirocyclic compound, for treatment or prevention of ILD, including PF and IPF. The agents can be combined with the spirocyclic compound in a single dosage form, or the agents can be administered simultaneously or sequentially in separate dosage forms.

The spirocyclic compound can be administered to patients (animals and humans) in need of such treatment in appropriate dosages that will provide prophylactic and/or therapeutic efficacy. The dose required for use in the treatment or prevention of any particular disease or disorder will typically vary from patient to patient depending on, for example, particular compound or composition selected, the route of administration, the nature of the condition being treated, the age and condition of the patient, concurrent medication or special diets then being followed by the patient, and other factors. The appropriate dosage can be determined by the treating physician.

The spirocyclic compound can be administered orally, subcutaneously, topically, parenterally, by inhalation spray or rectally in dosage unit formulations containing pharmaceutically acceptable carriers, adjuvants and vehicles. Parenteral administration can involve subcutaneous injections, intravenous or intramuscular injections or infusion techniques. Injectable liquids can include aqueous and/or organic components. Treatment duration can be as long as deemed necessary by a treating physician. The compositions can be administered as often as needed, e.g., one to four (or more) or more times per day. A treatment period can terminate when a desired result, for example, a particular therapeutic effect, is achieved. Or a treatment period can be continued indefinitely.

In some embodiments, the pharmaceutical compositions can be prepared as solid dosage forms for oral administration (e.g., capsules, tablets, pills, dragees, powders, granules and the like). A tablet can be prepared by compression or molding. Compressed tablets can include pharmaceutical excipients, such as binders, lubricants, chelants, sequestering agents, glidants, inert diluents, preservatives, disintegrants, or dispersing agents. Tablets and other solid dosage forms, such as capsules, pills, powders, and granules, can include coatings, such as enteric coatings.

Liquid dosage forms for oral administration can include, for example, vehicles such as pharmaceutically acceptable emulsions, microemulsions, solutions, suspensions, syrups and elixirs. The liquid vehicles can have aqueous and/or organic components. Suspensions can include one or more suspending agents. Examples of ingredients useful in liquid dosage forms can include one or more chelants, sequestering agents, viscosifiers, thickeners, penetration enhancers, solvents, diluents, and emulsifiers.

The amount of THP1 inhibitor to be administered will vary depending on factors such as the following: the THP1 inhibitor selected, method of administration, release profile, and composition formulation. Typically, for Compound A in an oral dosage form for ILD an/or IPF, a typical dosage will be about 1 mg/kg/day to about 50 mg/kg/day, and more typically from about 5 mg/kg/day to about 30 mg/kg/day, based on the weight of compound(s) (mg) and the patient (kg). Individual oral dosage forms typically have from about 50 mg to about 3000 mg of a spirocyclic compound and additional amounts of one or more pharmaceutically acceptable excipients. Other useful individual oral dosage forms can, by way of example, have spirocyclic compound in amounts of 100 mg, 150 mg, 200 mg, 250 mg, 300 mg, 350 mg, or 400 mg, 450 mg, 500 mg, 550 mg, 575 mg, 600 mg, 625 mg, 650 mg, 675 mg, 700 mg, 725 mg, 750 mg, 775 mg, or 800 mg. Other amounts between 50 mg to 3000 mg are possible, for example, from about 325 mg to about 475 mg, from about 350 mg to about 500 mg, from about 375 to about 525 mg, from about 400 mg to about 550 mg, from about 425 mg to about 575 mg, from about 450 mg to about 600 mg, from about 475 mg to about 625 mg, from about 500 mg to about 650 mg, from about 525 mg to about 675 mg, from about 550 mg to about 700 mg, from about 575 mg to about 725 mg, from about 600 mg to about 750 mg, from about 625 mg to about 775 mg, from about 650 mg to about 800 mg, from about 675 mg to about 825 mg, from about 700 mg to about 850 mg, from about 725 mg to about 875 mg, from about 750 mg to about 900 mg, from about 775 mg to about 925 mg, from about 800 mg to about 950 mg, from about 825 mg to about 975 mg, and from about 850 mg to about 1000 mg. A spirocyclic compound useful in the present invention is Compound A in crystalline Form 3.

Compositions for inhalation or insufflation include solutions and suspensions in pharmaceutically acceptable aqueous or organic solvents, or mixtures thereof, and powders. Liquid dosage forms for oral administration can include, for example, vehicles such as one or more pharmaceutically acceptable emulsions, microemulsions, solutions, suspensions, syrups and elixirs. The liquid vehicles can have aqueous and/or organic components. Suspensions can include one or more suspending agents.

Typically, for Compound A compositions for inhalation or insufflation for ILD and/or IPF, a typical dosage will be about 0.1 mg/kg/day to about 50 mg/kg/day based on the weight of compound(s) (mg) and the patient (kg).

Dosage forms for topical or transdermal administration of a subject composition include powders, sprays, ointments, pastes, creams, lotions, gels, solutions, and patches.

The spirocyclic compound of and compositions containing same can be administered by aerosol which can be administered, for example, by a sonic nebulizer.

Pharmaceutical compositions suitable for parenteral administration can include the spirocyclic compound together with one or more pharmaceutically acceptable sterile isotonic aqueous or non-aqueous solutions, dispersions, suspensions or emulsions.

Alternatively, the composition can be in the form of a sterile powder which can be reconstituted into a sterile injectable solutions or dispersion just prior to use.

Solid and liquid dosage forms can be formulated such that they conform to a desired release profile, e.g., immediate release, delayed release, and extended/sustained release.

It should be understood that the foregoing description is only illustrative of the present disclosure. Various alternatives and modifications can be devised by those skilled in the art without departing from the present disclosure. Accordingly, the present disclosure is intended to embrace all such alternatives, modifications and variances which fall within the scope of the appended claims.

What is claimed is:

1. A method of treating or preventing idiopathic pulmonary fibrosis in a patient comprising administering to the patient a therapeutically effective amount of the compound of the following formula: 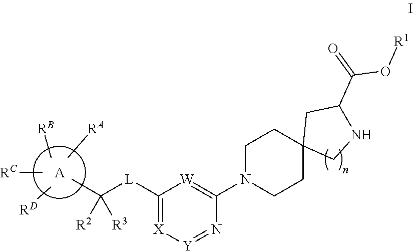
or a pharmaceutically acceptable salt thereof, wherein:
Ring A is C₃₋₁₀ cycloalkyl, C₆₋₁₀ aryl, 4 to 10-membered heterocycloalkyl, or 5 to 10-membered heteroaryl;
L is O or NR⁴;
W is N or CR⁵;
X is N or CR⁶;
Y is N or CR⁷;
wherein only one of X and Y is N;
R¹ is H, C₁₋₁₀ alkyl, C₃₋₁₀ cycloalkyl, phenyl, —(CR⁸R⁹)ₚOC(O)R¹⁰, —(CR⁸R⁹)ₚNR¹¹R¹², or —(CR⁸R⁹)ₚC(O)NR¹¹R¹², wherein said C₁₋₁₀ alkyl, C₃₋₁₀ cycloalkyl, and phenyl are each optionally substituted with 1, 2, 3, 4, or 5 substituents independently selected from F, Cl, Br, CN, C₁₋₄alkyl, and C₁₋₄ haloalkyl;
R² and R³ are each independently selected from H, C₁₋₄alkyl, and C₁₋₄ haloalkyl;
R⁴ is H or C₁₋₄alkyl;
R⁵ and R⁶ are each independently selected from H, halo, and C₁₋₄alkyl;
R⁷ is H, C₁₋₄alkyl, C₂₋₆ alkenyl, C₃₋₁₀ cycloalkyl, C₃₋₁₀ cycloalkyl-C₁₋₄ alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, (5-10 membered heteroaryl)-C₁₋₄alkyl, NR¹³R¹⁴, OR¹⁵, C(O)R¹⁶, S(O)_{q}R¹⁷, wherein said C₁₋₄alkyl, C₂₋₆ alkenyl, C₃₋₁₀ cycloalkyl, C₃₋₁₀ cycloalkyl-C₁₋₄ alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, and (5-10 membered heteroaryl)-C₁₋₄alkyl are each optionally substituted by 1, 2, or 3 substituents selected from halo, C₁₋₄alkyl, C₂₋₆ alkenyl, amino, C₁₋₄ alkylamino, C₂₋₈ dialkylamino, hydroxy, and C₁₋₄alkoxy;
R⁸ and R⁹ are each independently selected from H and C₁₋₄alkyl;
R¹⁰ is C₁₋₆alkyl optionally substituted by 1, 2 or 3 substituents independently selected from C₁₋₆ haloalkyl, C₃₋₁₀ cycloalkyl, OR^{a}, and NR^{c}R^{d};
R¹¹ and R¹² are each independently selected from H and C₁₋₆alkyl;
R¹³ is H or C₁₋₄alkyl;
R¹⁴ is H, C₁₋₄alkyl, C₃₋₇ cycloalkyl, C₃₋₇cycloalkyl-C₁₋₄alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, or (5-10 membered heteroaryl)-C₁₋₄alkyl, C(O)R^{b1}, C(O)OR^{a1}, C(O)NR^{c1}R^{d1}, S(O)R^{b1}, S(O)₂R^{b1}, or S(O)₂NR^{c1}R^{d1}, wherein said C₁₋₄alkyl, C₃₋₇ cycloalkyl, C₃₋₇cycloalkyl-C₁₋₄alkyl, C₆₋₁₀ aryl, C₆₋₁₀ aryl-C₁₋₄ alkyl, 4-10 membered heterocycloalkyl, (4-10 membered heterocycloalkyl)-C₁₋₄ alkyl, 5-10 membered heteroaryl, and (5-10 membered heteroaryl)-C₁₋₄alkyl are each optionally substituted by 1, 2, or 3 substituents independently selected from halo, C₁₋₄ alkyl, C₁₋₄ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; or
R¹³ and R¹⁴ together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};
R¹⁵ is H, C₁₋₄alkyl, C₃₋₇cycloalkyl, C₃₋₇cycloalkyl-C₁₋₄alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, or (5-10 membered heteroaryl)-C₁₋₄alkyl, wherein said C₁₋₄alkyl, C₃₋₇cycloalkyl, C₃₋₇ cycloalkyl-C₁₋₄alkyl, C₆₋₁₀ aryl, C₆₋₁₀ aryl-C₁₋₄alkyl, 4-10 membered heterocycloalkyl, (4-10 membered heterocycloalkyl)-C₁₋₄alkyl, 5-10 membered heteroaryl, and (5-10 membered heteroaryl)-C₁₋₄alkyl are each optionally substituted by 1, 2, or 3 substituents independently selected from halo, C₃₋₇cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};
R¹⁶ is C₁₋₄ alkyl or NR^{18a}R^{18b} wherein said C₁₋₄alkyl is optionally substituted by 1, 2, or 3 substituents independently selected from halo, C₃₋₇ cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};
R¹⁷ is C₁₋₄ alkyl, NR^{18a}R^{18b}, or OR^{18c}, wherein said C₁₋₄ alkyl is optionally substituted by 1, 2, or 3 substituents independently selected from halo, C₃₋₇ cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};
R^{18a} and R^{18b} are each independently selected from H and C₁₋₄ alkyl wherein said C₁₋₄ alkyl is optionally substituted by 1, 2, or 3 substituents independently selected from halo, C₃₋₇ cycloalkyl, 4-7 membered heterocycloalkyl, C₆₋₁₀ aryl, 5-6 membered heteroaryl, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c4}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1}; or
R^{18a} and R^{18b} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, S(O)R^{b1}, S(O)NR^{c1}R^{d1}, S(O)₂R^{b1}, NR^{c1}S(O)₂R^{b1}, NR^{c1}S(O)₂NR^{c1}R^{d1}, and S(O)₂NR^{c1}R^{d1};
R^{18c} is H, C₁₋₆ alkyl, C₃₋₁₀ cycloalkyl, C₃₋₇cycloalkyl-C₁₋₄ alkyl, C₆₋₁₀ aryl, C₆₋₁₀ aryl-C₁₋₄ alkyl, 4-10 membered heterocycloalkyl, (4-10 membered heterocycloalkyl)-C₁₋₄alkyl, 5-10 membered heteroaryl, or (5-10 membered heteroaryl)-C₁₋₄alkyl, wherein said C₁₋₆alkyl, C₃₋₇ cycloalkyl, C₃₋₁₀ cycloalkyl-C₁₋₄alkyl, C₆₋₁₀ aryl, C₆₋₁₀ aryl-C₁₋₄alkyl, 4-10 membered heterocycloalkyl, (4-10 membered heterocycloalkyl)-C₁₋₄ alkyl, 5-10 membered heteroaryl, and (5-10 membered heteroaryl)-C₁₋₄ alkyl are each optionally substituted by 1, 2, or 3 substituents independently selected from halo, C₁₋₄ alkyl, C₁₋₄ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R^{A} is H, Cy¹, halo, C₁₋₆ alkyl, C₂₋₆ alkenyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, or S(O)₂NR^{c2}R^{d2}, wherein said C₁₋₆ alkyl and C₂₋₆ alkenyl are each optionally substituted with 1, 2, 3, 4, or 5 substituents independently selected from Cy¹, halo, C₁₋₆ alkyl, C₂₋₆ alkenyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{B} is H, Cy², halo, C₁₋₆ alkyl, C₂₋₆ alkenyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, or S(O)₂NR^{c3}R^{d3}, wherein said C₁₋₆ alkyl and C₂₋₆ alkenyl are each optionally substituted with 1, 2, 3, 4, or 5 substituents independently selected from Cy², halo, C₁₋₆ alkyl, C₂₋₆ alkenyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c1}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
R^{C} and R^{D} are independently selected from H, halo, C₁₋₆ alkyl, C₂₋₆ alkenyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl and C₂₋₆ alkenyl are each optionally substituted with 1, 2, 3, 4, or 5 substituents independently selected from C₆₋₁₀ aryl, C₃₋₁₀ cycloalkyl, 5-10 membered heteroaryl, 4-10 membered heterocycloalkyl, halo, C₁₋₆ alkyl, C₂₋₆ alkenyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
Cy¹ and Cy² are each independently selected from C₆₋₁₀ aryl, C₃₋₁₀ cycloalkyl, 5-10 membered heteroaryl, and 4-10 membered heterocycloalkyl, each of which is optionally substituted by 1, 2, 3, 4, or 5 substituents independently selected from R^{Cy};
each R^{Cy} is independently selected from halo, C₁₋₆ alkyl, C₁₋₆ haloalkyl, C₂₋₆ alkenyl, C₆₋₁₀ aryl, C₃₋₁₀ cycloalkyl, 5-10 membered heteroaryl, 4-10 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₂₋₆ alkenyl C₆₋₁₀ aryl, C₃₋₁₀ cycloalkyl, 5-10 membered heteroaryl, and 4-10 membered heterocycloalkyl are each optionally substituted with 1, 2, 3, 4, or 5 substituents independently selected from halo, C₁₋₆ alkyl, CN, NO₂, R^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
each R^{a}, R^{a1}, R^{a2}, R^{a3}, R^{a4}, and R^{a5} is independently selected from H, C₁₋₆ alkyl, C₁₋₄ haloalkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, or (4-10 membered heterocycloalkyl)-C₁₋₄alkyl, wherein said C₁₋₆ alkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄alkyl, and (4-10 membered heterocycloalkyl)-C₁₋₄alkyl are each optionally substituted with 1, 2, 3, 4, or 5 substituents independently selected from C₁₋₄ alkyl, halo, CN, OR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
each R^{b1}, R^{b2}, R^{b3}, R^{b4}, and R^{b5} is independently selected from H, C₁₋₆ alkyl, C₁₋₄ haloalkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, or (4-10 membered heterocycloalkyl)-C₁₋₄alkyl, wherein said C₁₋₆ alkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄alkyl, and (4-10 membered heterocycloalkyl)-C₁₋₄ alkyl are each optionally substituted with 1, 2, 3, 4, or 5 substituents independently selected from C₁₋₄ alkyl, halo, CN, OR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
each R^{c}, R^{d}, R^{c1}, R^{d1}, R^{c2}, R^{d2}, R^{c3}, R^{d3}, R^{c4}, R^{d4}, R^{c5}, and R^{d5} is independently selected from H, C₁₋₆ alkyl, C₁₋₄ haloalkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄alkyl, or (4-10 membered heterocycloalkyl)-C₁₋₄alkyl, wherein said C₁₋₆ alkyl, C₂₋₆ alkenyl, C₆₋₁₀ aryl, C₃₋₁₀ cycloalkyl, 5-10 membered heteroaryl, 4-10 membered heterocycloalkyl, C₆₋₁₀ aryl-C₁₋₄alkyl, C₃₋₁₀ cycloalkyl-C₁₋₄alkyl, (5-10 membered heteroaryl)-C₁₋₄alkyl, and (4-10 membered heterocycloalkyl)-C₁₋₄alkyl are each optionally substituted with 1, 2, 3, 4, or 5 substituents independently selected from C₁₋₄ alkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or
any R^{c} and R^{d} together with the N atom to which they are attached form a 4-, 5-, 6-, or 7-membered heterocycloalkyl group optionally substituted with 1, 2, or 3 substituents independently selected from C₁₋₆ alkyl, C₃₋₇cycloalkyl, 4-7 membered heterocycloalkyl, C₆₋₁₀ aryl, 5-6 membered heteroaryl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or
any R^{c1} and R^{d1} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or
any R^{c2} and R^{d2} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, and 5-6 membered heteroaryl, C₁₋₆haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or
any R^{c3} and R^{d3} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, C₁₋₆haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or
any R^{c4} and R^{d4} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, C₁₋₆ haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇ cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}; or
any R^{c5} and R^{d5} together with the N atom to which they are attached form a 4-, 5-, 6-, or 7-membered heterocycloalkyl group optionally substituted with 1, 2, or 3 substituents independently selected from C₁₋₆ alkyl, C₃₋₇ cycloalkyl, 4-7 membered heterocycloalkyl, C₆₋₁₀ aryl, 5-6 membered heteroaryl, C₁₋₆haloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6}, wherein said C₁₋₆ alkyl, C₃₋₇cycloalkyl, 4-7 membered heterocycloalkyl, C₆₋₁₀ aryl, and 5-6 membered heteroaryl are each optionally substituted by 1, 2, or 3 substituents independently selected from halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
each R^{a6}, R^{b6}, R^{c6}, and R^{d6} is independently selected from H, C₁₋₄ alkyl, C₂₋₄ alkenyl, C₃₋₇ cycloalkyl, phenyl, 5-6 membered heteroaryl, and 4-7 membered heterocycloalkyl, wherein said C₁₋₄ alkyl, C₂₋₄ alkenyl, C₃₋₇ cycloalkyl, phenyl, 5-6 membered heteroaryl, and 4-7 membered heterocycloalkyl are each optionally substituted by 1, 2, or 3 substituents independently selected from OH, CN, amino, halo, C₁₋₄ alkyl, C₁₋₄alkoxy, C₁₋₄ alkylthio, C₁₋₄alkylamino, and di(C₁₋₄alkyl)amino;
n is 1 or 2;
p is 1, 2, or 3; and
q is 1 or 2;
wherein any aforementioned 4-10 or 4-7 membered heterocycloalkyl group optionally comprises 1, 2, or 3 oxo substituents, wherein each oxo substituent that is present is substituted on a ring-forming carbon, nitrogen, or sulfur atom of the 4-10 or 4-7 membered heterocycloalkyl group.

2. The method of claim 1, or a pharmaceutically acceptable salt thereof, having Formula VII: 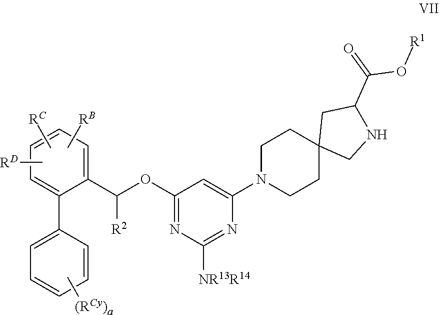
wherein a is 0, 1, 2, or 3.

3. The method of claim 1, wherein the compound or pharmaceutically acceptable salt thereof is administered orally, subcutaneously, topically, parenterally, by inhalant, by aerosol, or rectally.

4. The method of claim 1, wherein the compound or pharmaceutically acceptable salt thereof is administered orally by a dosage form selected from the group consisting of capsules, tablets, pills, dragees, powders, and granules.

5. The method of claim 3, wherein the compound or pharmaceutically acceptable salt thereof is administered one to four times per day.

6. The use of the compound or pharmaceutically acceptable salt thereof of claim 1 in the treatment of idiopathic pulmonary fibrosis.

7. The method of claim 1, wherein the compound is (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate.

8. The method of claim of 7 wherein the compound is in a substantially crystalline form.

9. The method of claim 8, wherein the compound is a crystalline polymorph having a XRPD plot corresponding to FIG. 3.

10. The method of claim 8, wherein the compound is a crystalline polymorph having a XRPD plot corresponding to Table 2.

11. The method of claim 1, wherein the compound is (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid.

12. The method of claim 11 wherein the compound is in a substantially crystalline form.

13. A method of treating or preventing interstitial lung disease in a patient, comprising: administering to said patient a therapeutically effective amount of a THP1 inhibitor.

14. The method of claim 13, wherein the THP1 inhibitor is a compound or pharmaceutically acceptable salt thereof used in method claim 1.

15. A method of treating or preventing pulmonary fibrosis in a patient, comprising: administering to said patient a therapeutically effective amount of a THP1 inhibitor.

16. The method of claim 15, wherein the THP1 inhibitor is a compound or pharmaceutically acceptable salt thereof used in method claim 1.
